(19) United States
(12) Patent Application Publication
(10) Pub. No.: US 2022/0362125 A1
(43) Pub. Date: Nov. 17, 2022

(54) COMPOSITION AND APPLICATION THEREOF IN PREPARATION OF SKIN CARE PRODUCTS FOR REGULATING SKIN BIORHYTHM

(71) Applicant: SYOUNG COSMETICS MANUFACTURING CO., LTD., Changsha, Hunan (CN)

(72) Inventors: Yuefeng DAI, Changsha, Hunan (CN); Shaowei YAN, Changsha, Hunan (CN); Guangwen HE, Changsha, Hunan (CN); Jingru QIAN, Changsha, Hunan (CN); Xiaoqun XU, Changsha, Hunan (CN)

(73) Assignee: SYOUNG COSMETICS MANUFACTURING CO., LTD., Changsha, Hunan (CN)

(21) Appl. No.: 17/764,184

(22) PCT Filed: Aug. 28, 2020
(86) PCT No.: PCT/CN2020/112021

§ 371 Date: Mar. 26, 2022

(30) Foreign Application Priority Data
Sep. 26, 2019 (CN) 201910919384.X

(51) Int. Cl. A61K 8/64; A61Q 19/00; A61K 8/44; A61K 8/49; A61K 8/60

(52) U.S. Cl. CPC A61K 8/64; A61Q 19/007; A61K 8/44; A61K 8/4946; A61K 8/606

(57) ABSTRACT

A skin care composition for regulating skin biorhythm, comprising: a skin conditioning agent A, a skin conditioning agent B, and adenosine. The skin conditioning agent A comprises glycoprotein, amino acid, a preservative, a stabilizing agent, and water, and the skin conditioning agent B comprises glutamine-based ethyl imidazole, a preservative, and water. The skin care composition can replenish moisture and promote cellular energy synthesis, and can solve the skin problem of people who stay up late.

This application claims the priority of Chinese Patent Application No. 201910919384.X, filed with the China National Intellectual Property Administration on Sep. 26, 2019, and titled with “COMPOSITION AND APPLICATION THEREOF IN PREPARATION OF SKIN CARE PRODUCTS FOR REGULATING SKIN BIORHYTHM”, which is hereby incorporated by reference in entirety.

### FIELD

The present disclosure relates to the technical field of skin care products, specifically to a composition and an application thereof in the preparation of skin care products for regulating skin biorhythm.

### BACKGROUND

The organism is supervised by the internal clock-biorhythm-24 hours, and the periodic expression of circadian rhythm genes is under control. The purpose of this periodic activation of circadian rhythm genes is to actively adapt to changes in metabolism and protein synthesis function brought about by daily environmental changes. Sunlight exposure is one of the activators synchronizing biorhythms, so the periodicity of circadian rhythm gene expression will be affected by the environment. The retina and skin are the main receptors for sunlight.

The physiological activity of skin tissue is strictly controlled by biorhythms. During the day, the skin cells are mainly dedicated to strengthening the skin's natural defenses (resistance to UV, anti-pollution, etc.), and focus on regeneration and metabolism at night. Studies have found that biological clock genes of skin, such as CLOCK and PERIOD-1, are associated with the proliferation of cells (keratinocytes, fibroblasts and melanocytes).

Aging, changes in seasons and sun exposure, fatigue, tension, insomnia, jet lag, etc. can all cause small changes in biorhythms, but these changes can be accumulated. These irregular biorhythm changes cause the periodic activation of circadian rhythm genes to be changed, leading to changes in skin physiological metabolism.

Disordered biorhythms are one of the reasons that induce premature skin aging. When the physiological activity of the skin does not adapt to changes in environmental conditions, it may cause that the skin's natural defense capabilities weaken, the cell regeneration process at night is slow, and the biotransformation efficiency of vitamin D is reduced (Vitamin D is involved in calcium absorption, and the level of calcium affects the hydration capacity of the skin). However, there are few types of skin care products for the regulation of skin biorhythm, and the mechanism is not clear.

### SUMMARY

In view of this, the technical problem to be solved by the present disclosure is to provide a composition and an application thereof in the preparation of skin care products for regulating skin biorhythm. The composition of the present disclosure has good moisturizing and hydrating effects, and can promote cell energy synthesis.

The composition provided by the present disclosure comprises the following components in parts by mass:

| | |
|---|---|
| skin conditioner A | 0.2~1.0 parts; |
| skin conditioner B | 0.2~2.0 parts; |
| adenosine | 0.1~2.0 parts; |

wherein the skin conditioner A comprises the following in mass fraction:

| | |
|---|---|
| yeast glycoprotein | 3%; |
| glutamic acid | 3%; |
| valine | 0.55%; |
| threonine | 0.55%; |
| preservative | 1.1%; |
| stabilizer | 0.05% |
| and the balance is water; | |

the skin conditioner B comprises the following in mass fraction:

| | |
|---|---|
| glutamylamidoethyl imidazole | 1% |
| preservative | 0.4% |
| and the balance is water. | |

The preservative in the skin conditioner A is phenoxyethanol and ethylhexylglycerol, wherein a mass ratio of phenoxyethanol and ethylhexylglycerol is 1:1;

the stabilizer in the skin conditioner A is sodium metabisulfite;

the preservative in the skin conditioner B is sorbic acid and phenoxyethanol, wherein a mass ratio of sorbic acid and phenoxyethanol is 1:3.

The composition provided by the present disclosure comprises the following components in parts by mass:

| | |
|---|---|
| skin conditioner A | 0.5 parts; |
| skin conditioner B | 0.5 parts; |
| adenosine | 0.2 parts. |

The composition provided by the present disclosure comprises the following components in parts by mass:

| | |
|---|---|
| skin conditioner A | 0.2 parts; |
| skin conditioner B | 0.2 parts; |
| adenosine | 0.1 parts. |

The composition provided by the present disclosure comprises the following components in parts by mass:

| | |
|---|---|
| skin conditioner A | 1.0 parts; |
| skin conditioner B | 2.0 parts; |
| adenosine | 2.0 parts. |

The present disclosure takes the state of the skin staying up late as a research object, and takes the biorhythm mechanism of skin as a background. Firstly, the biorhythm peptide is used to regulate the biological clock genes of skin. When the body's biological clock is irregular, the skin's barrier is easily damaged, and epidermal keratinocytes are the only cells in the body that have a complete metabolic pathway of vitamin D. When the barrier of skin is damaged, that is, when the epidermis is damaged, keratinocytes are unable to synthesize vitamin D normally. The active ingredients contained in skin conditioner B can regulate the expression of biological clock genes, promote the repair and proliferation of keratinocytes, promote the synthesis of vitamin D, and activate the conversion of vitamin D in order to activate the barrier capacity of skin. In addition, from the perspective of providing more energy to skin for skin repair, through the regulation of biorhythms, skin conditioner A rich in glycoproteins, glutamic acid, valine, and threonine is used to promote sugar degradation and mitochondrial respiration to ensure that the ATPs of skin cells are at a high level throughout the day, such that enough energy is supplied to cells during the day to resist external unfavorable factors, and there is sufficient energy supply when skin cells need to be repaired at night. Further, the additional addition of adenosine directly supplements the energy required by the cells. The present disclosure aims at the problem of the skin staying up late, starting from the three dimensions of biological clock gene regulation+stimulation of cell own energy+extra energy supplementation, and develops a composition with strong care for the skin staying up late. The components in the composition of the present disclosure cooperate with each other, promote each other, and play a good synergistic effect.

Use of the composition of the present disclosure in the preparation of skin care product that regulates skin biorhythm.

The skin care product has functions including moisturizing and hydrating, and promoting cell energy synthesis.

The present disclosure also provides a skin care product for regulating skin biorhythm, comprising the composition of the present disclosure.

In the skin care product of the present disclosure, a mass fraction of the composition is 0.5%˜5%.

In some particular embodiments, the mass fraction of the composition is 0.5%, 1.2%, or 5%.

The skin care product of the present disclosure comprises:

| | |
|---|---|
| the composition of the present disclosure | 0.5%~5%; |
| humectant | 9%; |
| penetration enhancer | 1%; |
| keratin softener | 0.4%; |
| preservative | 1%; |
| and the balance is water. | |

In the skin care product of the present disclosure,

the humectant is glycerol and 1,3-butanediol, wherein a mass ratio of glycerol and 1,3-butanediol is 1:1;

the penetration enhancer is pentanediol;

the keratin softener is hydroxyethylpiperazine ethane sulfonic acid; and

the preservative is PHL.

The skin care product of the present disclosure consists of the following components in mass fraction:

| | |
|---|---|
| skin conditioner A | 0.2%~1.0%; |
| skin conditioner B | 0.2%~2.0%; |
| adenosine | 0.1%~2.0%; |
| glycerol | 8%; |
| 1,3 butanediol | 1%; |
| pentanediol | 1%; |
| hydroxyethylpiperazine ethane sulfonic acid | 0.4%; |
| PHL | 1%; |
| and the balance is water; | |

wherein the skin conditioner A comprises the following in mass fraction:

| | |
|---|---|
| yeast glycoprotein | 3%; |
| glutamic acid | 3%; |
| valine | 0.55%; |
| threonine | 0.55%; |
| preservative | 1.1%; |
| stabilizer | 0.05%; |
| and the balance is water; | |

the skin conditioner B comprises the following in mass fraction:

| | |
|---|---|
| glutamylamidoethyl imidazole | 1% |
| preservative | 0.4% |
| and the balance is water. | |

In some embodiments, the cosmetic consists of the following components:

| | |
|---|---|
| skin conditioner A | 0.5%; |
| skin conditioner B | 0.5%; |
| adenosine | 0.2%; |
| glycerol | 8%; |
| 1,3 butanediol | 1%; |
| pentanediol | 1%; |
| hydroxyethylpiperazine ethane sulfonic acid | 0.4%; |
| PHL | 1% |
| and the balance is water; | |

In some embodiments, the cosmetic consists of the following components:

| | |
|---|---|
| skin conditioner A | 0.2%; |
| skin conditioner B | 0.2%; |
| adenosine | 0.1%; |
| glycerol | 8%; |
| 1,3 butanediol | 1%; |
| pentanediol | 1%; |
| hydroxyethylpiperazine ethane sulfonic acid | 0.4%; |
| PHL | 1%; |
| and the balance is water; | |

In some embodiments, the cosmetic consists of the following components:

| | |
|---|---|
| skin conditioner A | 1.0%; |
| skin conditioner B | 2.0%; |
| adenosine | 2.0%; |
| glycerol | 8%; |
| 1,3 butanediol | 1%; |
| pentanediol | 1%; |
| hydroxyethylpiperazine ethane sulfonic acid | 0.4%; |
| PHL | 1%; |
| and the balance is water; | |

The skin care product of the present disclosure includes lotions, emulsions, creams, face masks, essences, gels and the like.

The method for preparing the skin care product of the present disclosure comprises:

adding hydroxyethylpiperazine ethane sulfonic acid and adenosine to deionized water, heating to 75° C.˜80° C., and stirring to dissolve at 250˜300 r/min for 20 min;

adding glycerol, 1,3-butanediol and pentanediol, controlling a temperature at 65˜75° C., and stirring to dissolve at 300 r/min for 15 min; and

adding the skin conditioner A, the skin conditioner B and PHL, controlling a temperature at 35˜45° C., stirring at 100 r/min for 15 min, and then cooling to room temperature to prepare a skin care product.

The composition provided by the present disclosure comprises adenosine, skin conditioner A containing glycoproteins and amino acids, and skin conditioner B containing glutamylamidoethyl imidazole. Each component cooperates with each other and promotes each other, so as to achieve good moisturizing and hydrating, and promote cell energy synthesis. The use of the composition in the preparation of cosmetics can improve a series of skin problems of people who stay up late.

### BRIEF DESCRIPTION OF DRAWINGS

FIG. 1 The influence of each sample on the energy synthesis of cells;

FIG. 2 Instant hydrating effect of each sample;

FIG. 3 Long-term hydrating effect of each sample;

FIG. 4 The short-term effects of each sample on the loss of skin water;

FIG. 5 The long-term effect of each sample on the loss of skin water.

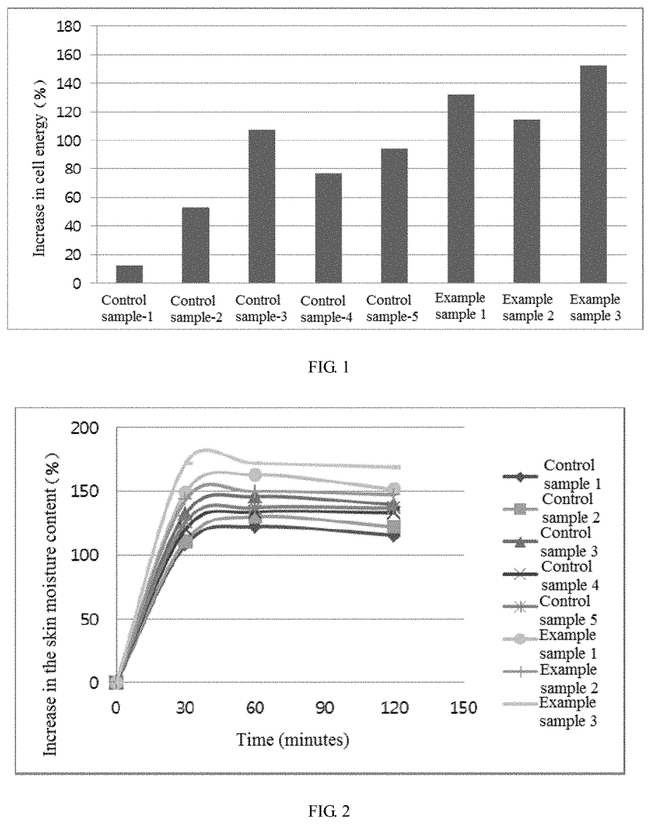

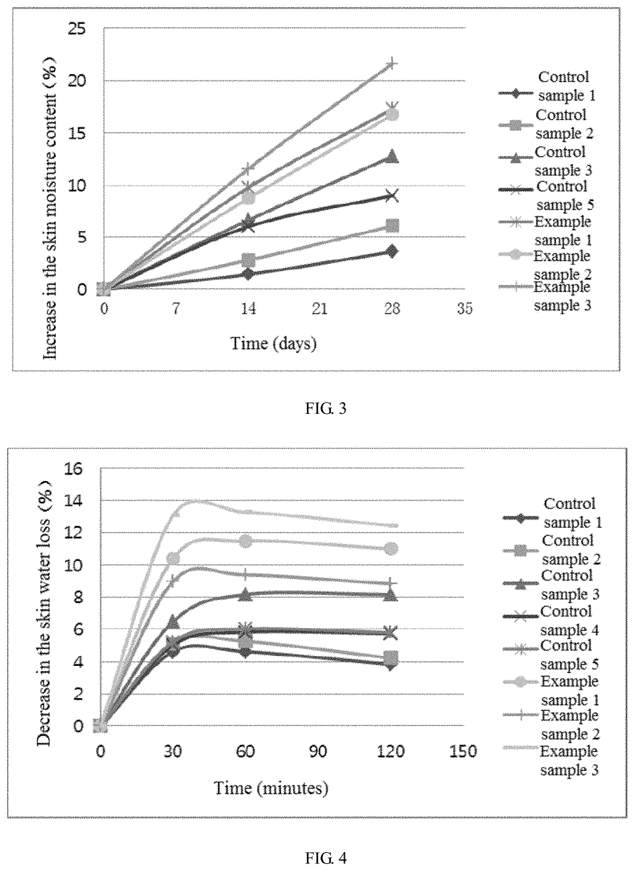

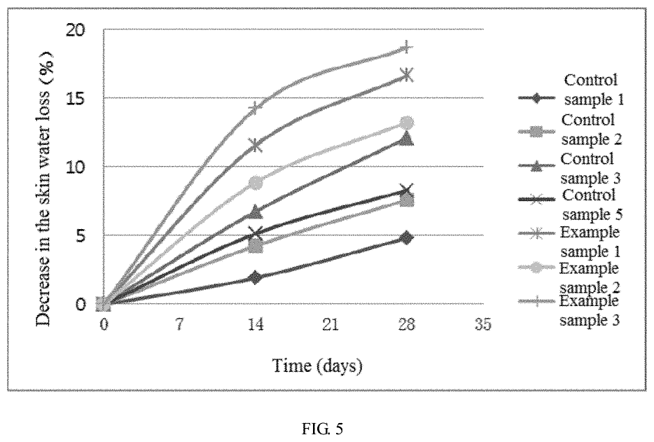

### DETAILED DESCRIPTION

The present disclosure provides a composition and an application thereof in the preparation of skin care products for regulating skin biorhythm. Those skilled in the art can learn from the disclosure and appropriately improve the process parameters. In particular, it should be noted that all similar substitutions and modifications will be obvious to those skilled in the art, which are all considered to be included in the present disclosure. The method and application of the present disclosure have been described through the preferred embodiments. It is obvious that relevant persons may modify or appropriately change and combine the methods and applications described herein without departing from the content, spirit and scope of the present disclosure in order to realize and apply the techniques of the present disclosure.

The reagents and materials used in the present disclosure are all common commercially available products, all of which can be purchased in the market.

In the following examples, the composition of the skin conditioner A used is:

| | |
|---|---|
| Glycoprotein | 3%; |
| Glutamic acid | 3%; |
| Valine | 0.55%; |
| Threonine | 0.55%; |
| Phenoxyethanol | 0.55%; |
| Ethylhexylglycerol | 0.55%; |
| Sodium metabisulfite | 0.05%; |
| Water | 91.75%. |

The composition of the skin conditioner B used is:

| | |
|---|---|
| Glutamylamidoethyl imidazole | 1%; |
| Sorbic acid | 0.1%; |
| Phenoxyethanol | 0.3%; |
| Water | 98.6%. |

Wherein, the structural formula of glutamylamidoethyl imidazole is:

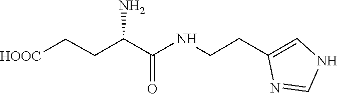 [SMILES: N[C@@H](CCC(=O)O)C(=O)NCCc1c[nH]cn1]

In the embodiment of the present disclosure, the skin conditioner A and skin conditioner B used are all purchased from the market, and the trade names are REVITALIN® PF and CHRONOCYCLIN® in sequence. The present disclosure will be further explained below in conjunction with examples.

### Examples

The formulas of each group are shown in Table 1:

TABLE 1
Formula content of each group (%)

| | Group | | | | | | | |
|---|---|---|---|---|---|---|---|---|
| | Control | Control | Control | Control | Control | Example | Example | Example |
| Component | sample 1 | sample 2 | sample 3 | sample 4 | sample 5 | 1 | 2 | 3 |
| Glycerol | 8.0 | 8.0 | 8.0 | 8.0 | 8.0 | 8.0 | 8.0 | 8.0 |
| 1,3 Butanediol | 1.0 | 1.0 | 1.0 | 1.0 | 1.0 | 1.0 | 1.0 | 1.0 |
| Pentanediol | 1.0 | 1.0 | 1.0 | 1.0 | 1.0 | 1.0 | 1.0 | 1.0 |
| Hydroxyethylpiperazine | 0.4 | 0.4 | 0.4 | 0.4 | 0.4 | 0.4 | 0.4 | 0.4 |
| ethane sulfonic acid | | | | | | | | |
| PHL | 1.0 | 1.0 | 1.0 | 1.0 | 1.0 | 1.0 | 1.0 | 1.0 |
| Skin conditioner B | — | 0.5 | 0.5 | — | 0.1 | 0.5 | 0.2 | 1.0 |
| Skin conditioner A | — | — | 0.5 | — | 0.1 | 0.5 | 0.2 | 2.0 |
| Adenosine | — | — | — | 1.0 | 1.0 | 0.2 | 0.1 | 2.0 |
| Water | 88.6 | 88.1 | 87.6 | 87.6 | 87.4 | 87.4 | 88.1 | 83.6 |

Hydroxyethylpiperazine sulfonic acid and adenosine were weighed according to the formula. They were added to deionized water. The system was heated to 75-80° C., and stirred at 250-300 r/min for 20 min until dissolved and clear.

Glycerol, 1,3-butanediol, and pentanediol were added according to the formula. The temperature was controlled at 65-75° C., and the system was stirred at 300 r/min for 15 min until all the additives were dissolved.

Skin conditioner A, skin conditioner B and PHIL were weighed according to the formula. The temperature was controlled at 35-45° C. The system was stirred at 100 r/min for 15 min, and allowed to stand to return to room temperature.

### Efficacy Evaluation

Epidermal keratinocytes are the only cells in the body that have a complete metabolic pathway of vitamin D. When the barrier of skin is damaged, that is, when the epidermis is damaged, keratinocytes are unable to synthesize vitamin D normally. Vitamin D is converted from 7-dehydrocholesterol in the epidermal tissue, and the epidermis also contains mitochondria CYP27A1 and CYP27B1. 1,2(OH)₂D has the effect of promoting the differentiation of epidermal keratinocytes, and this effect is completed by phospholipase C-γ1 (PLC-γ1). The 1,2(OH)₂D produced by epidermal keratinocytes co-regulates cell differentiation with calcium. 1,2(OH)₂D increases the expression of CaR, which increases the sensitivity of cells to calcium; and increases PLC-γ1 to increase the intracellular calcium ion concentration. Calcium ions have a regulatory effect on the formation of the epidermal barrier function. Applying a negative charge on the surface of the skin will increase the concentration of magnesium and calcium in the upper part of the epidermis, and increase the concentration gradient of calcium ions, which thereby enhances the barrier function of the epidermis. In addition, 1,2(OH)₂D directly stimulates the transcription of involucrin in keratinocytes. 1,2(OH)₂D can also promote the expression of bacteriostatic peptides in keratinocytes, thereby resisting skin bacterial infections. Vitamin D plays an important role in the skin barrier mainly by promoting the synthesis of filaggrin, antibacterial peptides and other proteins and regulating the proliferation and differentiation of keratinocytes. The present disclosure measures the efficacy of the composition from three aspects: cell energy change, hydrating effect, and loss of skin water.

### 1) Increase in Cell Energy

Adenosine 5′-triphosphate (ATP) is the most basic carrier for energy conversion in organisms, and changes in its content are directly related to the energy metabolism of various organs. As the most important energy molecule, ATP plays an important role in various physiological and pathological processes of cells. Changes in ATP levels can affect cell function. In general, when cells are in apoptosis, necrosis, or in some toxic state, ATP levels will drop. The decrease of ATP level during apoptosis usually coincides with the decrease of mitochondrial membrane potential. The ATP content detection kit can be used to detect ATP levels in cells.

Experimental instruments: microplate reader, water bath, pipette, centrifuge, 96-well plate, and ATP content detection kit.

Experimental materials: keratinocytes, DMEM, FBS, and DPBS.

Experimental Method:

1. After the keratinocytes were digested, DMEM medium was used to disperse the cells, and a hemacytometer was used to count the cells. Then DMEM was used to dilute the cells to a concentration of 5×10⁴ cells/ml.

2. The diluted cell solution was inoculated into petri dishes.

3. Incubation was performed for 24 hours in a 37° C., 5% CO₂ incubator.

4. Preparation of the sample to be tested: The sample to be tested was diluted with DMEM medium to a concentration of 0.1% after dilution, and 10 ml of each sample was prepared.

5. After cultured for 24 hours, the cells were observed whether they fully adhered to grow. If the cells fully adhered, the original medium was removed and the cells were washed with DPBS.

6. After the DPBS was removed, the previously prepared medium containing 0.1% of the sample to be tested was added.

7. After the sample was added, they were placed in a 37° C., 5% CO₂ incubator for 48 hours of culture.
Collection and treatment of keratinocytes: After the cells were collected, they were centrifuged to remove the medium, added with 0.5 ml of double distilled water, and mixed well. The cell aqueous solution was placed in boiling water, heated for 10 minutes, and vortexed for 1 minute to mix well. Centrifugation was performed at 4000 rpm for 10 minutes. The supernatant was taken out for testing.
The specific reagents and operation steps are as follows:

Experimental Reagents:

Reagent 1: Substrate liquid 1 was powder. It was added with 10 ml of distilled water to be dissolved when in use, and heated to be dissolved in boiling water.

Reagent 2: Substrate liquid 2 was liquid. It was used directly.

Reagent 3: Accelerator. The diluent was added to the powder to prepare a solution when in use.

Reagent 4: Precipitant

Reagent 5: Chromogenic agent. Liquid A was added to liquid B when in use, and they were mixed well for later use.

Reagent 6: Terminating agent

ATP standard: 1 mM ATP standard solution was prepared with DDW.

Experimental Method:

Preparation of Detection System:

TABLE 2
Detection system

| Reagent name (μL) | Detection tube | Control tube | Standard tube | Blank tube |
|---|---|---|---|---|
| Sample | 30 | 30 | — | — |
| 1 mM standard solution | — | — | 30 | 30 |
| Reagent 1 | 100 | 100 | 100 | 100 |
| Reagent 2 | 200 | 200 | 200 | 200 |
| Reagent 3 | 30 | — | 30 | — |
| Distilled water | — | 30 | — | 30 |

Mixing well. Reacting for 30 min at 37° C.

| | | | | |
|---|---|---|---|---|
| Reagent 4 | 50 | 50 | 50 | 50 |

Mixing well, centrifuging at 4000 rpm for 5 min, and taking 300 μL of the supernatant for determination

| | | | | |
|---|---|---|---|---|
| Sample supernatant | 300 | 300 | 300 | 300 |
| Reagent 5 | 500 | 500 | 500 | 500 |

Mixing well and standing for 2 min at room temperature

| | | | | |
|---|---|---|---|---|
| Reagent 6 | 500 | 500 | 500 | 500 |

The system was mixed well, and allowed to stand at room temperature to react for 5 minutes. 200 μL of sample from each detection tube, control tube, standard tube and blank tube was transferred to a 96-well plate, and the absorbance at 630 nm wavelength was tested.

Therefore, the ATP content of each test sample cell can be obtained as: ATP content (μmol)=[(A detection tube−A control tube)/(A standard tube−A blank tube)]×concentration of the standard×dilution factor
The increase in cell energy of each test sample:
ATP Increase (%)=(ATPₙ−ATP₀)/ATP₀×100% (where ATPₙ is the cell energy of each test sample, and ATP₀ is the cell energy of the blank sample)

Experimental Results:

TABLE 3
Energy synthesis of cells promoted by the patent combination

| | Increase in cell |
|---|---|
| | energy (%) |
| Control sample-1 | 12.47 |
| Control sample-2 | 53.20 |
| Control sample-3 | 107.65 |
| Control sample-5 | 76.82 |
| Control sample-4 (inappropriate) | 94.35 |
| Example sample-1 (patent sample) | 132.24 |
| Example sample-2 (lower limit sample) | 114.77 |
| Example sample-3 (upper limit sample) | 152.42 |

Experimental results: According to the results of the cell energy test, the control sample-1 had almost no effect on promoting cell energy growth; and the example samples 1˜3 can increase the cell energy by 114.77%˜152.42%. The increase of cell energy helps to repair the damaged skin condition. After statistical analysis:

Compared with Comparative Example 1, Examples 1˜3 can significantly increase cell energy, indicating that the composition of the present disclosure had a significant effect in improving cell energy; p<0.05;

Compared with Comparative Examples 2˜4, Examples 1˜3 had more significant effects in improving cell energy, indicating that the composition of the present disclosure was more reasonable in composition, each component was indispensable, and they cooperated with each other to produce a significant synergistic effect;

Compared with Comparative Example 5, Examples 1˜3 had more significant effects in improving cell energy, indicating that the composition of the present disclosure has a better effect in the same content of active ingredients, indicating that the composition of the present disclosure had a reasonable and appropriate ratio.

Among the samples, Example 3 had the most significant effect, indicating that the ratio and concentration were the most suitable. The effect of the obtained sample was significantly different from that of the other groups, p<0.05.

### 2) Instant and Long-Term Hydrating Effect on Skin

Test instrument: German CK company multifunctional skin tester, model Corneometer CM825 MDD,
Test principle: The capacitance method was used to test the moisture content of the stratum corneum of human skin. Its principle is based on the significant difference in dielectric constant between water and other substances. For different skin moisture content, the measured skin capacitance values are different. The observation parameters can represent the skin moisture value.
Test environment: In the test environment, the temperature was 22±1° C., the humidity was 50±5%, and real-time dynamic detection was performed;
Test volunteers: At least 30 effective volunteers, aged between 16-65 years old (except for pregnant or lactating women); the basic value of the capacitance method for skin moisture determination at the forearm test area being between 15-100; those who have no serious system disease, no immunodeficiency or autoimmune disease; those who have no previous history of severe allergies to skin care cosmetics; those who have not used hormone drugs and immunosuppressants in the past month; those who have not participated in other clinical trials; those who use the test drug according to the regulations and have complete information; all volunteers should fill out an informed consent form before the test.
Test steps:

Preparation before the test: No products (cosmetics or topical drugs) were used in the test site for 2-3 days. Before the experiment, subjects needed to agree to clean the inner forearms of their hands and let them air dry naturally. After cleaning, the measurement area on the inner forearms of the subject's hands was marked. Before the formal test, they should sit quietly in a room that met the standard for at least 30 min, without drinking water, with the forearms exposed and placed in the test state, and stay relaxed.

Test process: In the experiment, 3×3 cm² test areas were marked on the inner sides of the left and right arms, and multiple areas can be marked on the same arm, with an interval of 1 cm. The test product and the blank control were randomly distributed on the left and right arms. The probe Corneometer CM825 MDD was used to measure the skin moisture content of the test area and the control area. Each area was measured 15 times in parallel. First the blank value of each test area was measured, and then the sample was injected into the mask cloth at the amount of 0.072 ml sample/cm², which was applied to the test area for 20 minutes. After that, the mask cloth was taken off, and the skin moisture content of the area was tested after 10 minutes, which was the skin moisture content at 30 minutes. After that, the skin moisture content of the test area and the blank control area were measured at 1 hour and 2 hours respectively (measured at this time during verification), and the test on the same volunteer was completed by the same measurement staff.

For the long-term hydrating test, the subjects applied the test sample in the same area every day according to the above test method, and tested the skin moisture content on the 14th and 28th day without applying the test sample.

Test data: According to the design of the experiment, the skin moisture content of each time period was measured, and the increase in the skin moisture content at each time point was calculated.

$$Increase in the skin moisture content ( \% ) = \frac{{M}_{1} - {M}_{0}}{{M}_{0}} \times 100 \%$$

Experimental Results:

2.1) Instant Hydrating Results

TABLE 4
Instant hydrating effect

| | Increase in skin moisture content (%) | | | |
|---|---|---|---|---|
| Time (minutes) | 0 | 30 | 60 | 120 |
| Control sample-1 | 0.0 | 108.1 | 122.3 | 115.6 |
| Control sample-2 | 0.0 | 111.0 | 130.1 | 122.2 |
| Control sample-3 | 0.0 | 132.6 | 145.8 | 139.7 |
| Control sample-4 | 0.0 | 120.3 | 133.7 | 133.0 |
| Control sample-5 | 0.0 | 125.9 | 137.2 | 136.9 |
| (inappropriate) | | | | |
| Example sample-1 | 0.0 | 149.3 | 163 | 151.5 |
| (patent sample) | | | | |
| Example sample-2 | 0.0 | 143.6 | 149.9 | 147.2 |
| (lower limit sample) | | | | |
| Example sample-3 | 0.0 | 172.1 | 171.9 | 168.8 |
| (upper limit sample) | | | | |

Result: From the perspective of the instant hydrating effect, control sample-1 as the basic formula had a certain hydrating effect, and the increase in moisture content was 108.1%. The skin moisture content of example sample-1 increased to 149.3% at 30 minutes, and the hydrating effect was still about 151.5% at 120 minutes. The instant hydrating effect was obvious and lasted for a long time.

2.2) Long-Term Hydrating Effect

TABLE 5
Long-term hydrating effect

| | Increase in skin moisture content (%) | | |
|---|---|---|---|
| Time (days) | 0 | 14 | 28 |
| Control sample-1 | 0.00 | 1.46 | 3.62 |
| Control sample-2 | 0.00 | 2.81 | 6.09 |
| Control sample-3 | 0.00 | 6.64 | 12.73 |
| Control sample-4 | — | — | — |
| Control sample-5 | 0.00 | 6.02 | 8.98 |
| (inappropriate) | | | |
| Example sample-1 | 0.00 | 9.75 | 17.27 |
| (patent sample) | | | |
| Example sample-2 | 0.00 | 8.73 | 16.72 |
| (lower limit sample) | | | |
| Example sample-3 | 0.00 | 11.54 | 21.60 |
| (upper limit sample) | | | |

Result: From the perspective of the long-term hydrating effect, if the patent sample was used continuously, the skin moisture content would have a process of growth. At 28 days, the skin moisture content increased by about 17.27%. At the same time, it also reflected that the barrier function of skin had been repaired and the skin moisture content of had been improved.

Compared with Comparative Example 1, Examples 1˜3 can significantly improve the hydrating effect (long-term, instant), indicating that the composition of the present disclosure had a significant effect in the ability of hydrating (long-term, instant); p<0.05;

Compared with Comparative Examples 2˜4, Examples 1˜3 had more significant effects in hydrating (long-term, instant), indicating that the composition of the present disclosure was more reasonable in composition, each component was indispensable, and they cooperated with each other to produce a significant synergistic effect;

Compared with Comparative Example 5, Examples 1˜3 had more significant effects in hydrating (long-term, instant), indicating that the composition of the present disclosure has a better effect in the same content of active ingredients, indicating that the composition of the present disclosure had a reasonable and appropriate ratio.

Among the samples, the effect of Example 3 was the most significant, indicating that the ratio and concentration were the most suitable. The effect of the obtained sample was significantly different from that of the other groups, p<0.05.

### 3) Skin Barrier Repair Effect (TEWL Value)

Test instrument: German CK company multifunctional skin tester, probe model Tewameter TM300,
Test principle: FICK's law of diffusion: dm/dt=D·A·dp/dx. Two sets of temperature and humidity sensors were used to measure the water vapor pressure gradient formed by the water loss of the stratum corneum at different bright spots near the epidermis (within about 1 cm), through which the transepidermal water loss was directly measured. The IEWL value is an important indicator of the quality of skin barrier. The lower the TEWL value of the skin, the better the barrier function of the skin, and vice versa.
Test environment: In the test environment, the temperature was 22±1° C., the humidity was 50±5%, and real-time dynamic detection was performed;
Test volunteers: At least 30 effective volunteers, aged between 16-65 years old (except for pregnant or lactating women); those who have no serious system disease, no immunodeficiency or autoimmune disease; those who have no previous history of severe allergies to skin care cosmetics; those who have not used hormone drugs and immunosuppressants in the past month; those who have not participated in other clinical trials; those who use the test drug according to the regulations and have complete information; all volunteers should fill out an informed consent form before the test.
Test steps:

Preparation before the test: No products (cosmetics or topical drugs) were used in the test site for 2-3 days. Before the experiment, subjects needed to agree to clean the inner forearms of their hands and let them air dry naturally. After cleaning, the measurement area on the inner forearms of the subject's hands was marked. Before the formal test, they should sit quietly in a room that met the standard for at least 30 min, without drinking water, with the forearms exposed and placed in the test state, and stay relaxed.

Test process: In the experiment, 3×3 cm² test areas were marked on the inner sides of the left and right arms, and multiple areas can be marked on the same arm, with an interval of 1 cm. The test product and the blank control were randomly distributed on the left and right arms. The probe Tewameter TM300 was used to measure the skin water loss of the test area and the control area. Each area was measured 15 times in parallel. First the blank value of each test area was measured, and then the sample was injected into the mask cloth at the amount of 0.072 ml sample/cm², which was applied to the test area for 20 minutes. After that, the mask cloth was taken off, and the skin water loss of the area was tested after 10 minutes, which was the skin water loss at 30 minutes. After that, the skin water loss of the test area and the blank control area were measured at 1 hour and 2 hours respectively, and the test on the same volunteer was completed by the same measurement staff.

For the long-term barrier repair test, the subjects applied the test sample in the same area every day according to the above test method, and tested the skin water loss TEWL on the 14th and 28th day without applying the test sample.

Test data: According to the design of the experiment, the skin water loss of each time period was measured, and the decrease in the skin water loss at each time point was calculated. The greater the decrease in the skin water loss, the better the effect of skin barrier repair.

$$Decrease in the skin water loss ( \% ) = \frac{( {T}_{0} - {T}_{1} )}{{T}_{0}} \times 100 \%$$

Experimental Results:

3.1) Instant Barrier Repair Effect

TABLE 6
Decrease in the instant skin water loss

| | Decrease in the skin water loss (%) | | | |
|---|---|---|---|---|
| Time (min) | 0 | 30 | 60 | 120 |
| Control sample-1 | 0.00 | 4.62 | 4.62 | 3.84 |
| Control sample-2 | 0.00 | 5.11 | 5.26 | 4.22 |
| Control sample-3 | 0.00 | 6.49 | 8.16 | 8.15 |
| Control sample-4 | 0.00 | 5.02 | 5.83 | 5.74 |
| Control sample-5 | 0.00 | 5.22 | 6.04 | 5.80 |
| (inappropriate) | | | | |
| Example sample-1 | 0.00 | 10.41 | 11.48 | 11.00 |
| (patent sample) | | | | |
| Example sample-2 | 0.00 | 8.97 | 9.38 | 8.83 |
| (lower limit sample) | | | | |
| Example sample-3 | 0.00 | 13.07 | 13.27 | 12.45 |
| (upper limit sample) | | | | |

Results: From the perspective of the decrease in the skin water loss, control sample-1 as the basic sample had a certain effect of reducing the skin water loss. At 30 minutes, the decrease in the skin water loss was 4.62%, and the decrease in the skin water loss of the patent sample reached 10.41%, indicating a significantly reduced skin water loss, which reflected the effect of repairing the skin barrier.

3.2) Long-Term Barrier Repair Effect

TABLE 7
Decrease in long-term skin water loss

| | Decrease in the skin water loss (%) | | |
|---|---|---|---|
| Time (days) | 0 | 14 | 28 |
| Control sample-1 | 0.00 | 1.93 | 4.84 |
| Control sample-2 | 0.00 | 4.26 | 7.60 |
| Control sample-3 | 0.00 | 6.75 | 12.11 |
| Control sample-4 | — | — | — |
| Control sample-5 | 0.00 | 5.14 | 8.31 |
| (inappropriate) | | | |
| Example sample-1 | 0.00 | 11.57 | 16.64 |
| (patent sample) | | | |
| Example sample-2 | 0.00 | 8.86 | 13.24 |
| (lower limit sample) | | | |
| Example sample-3 | 0.00 | 14.24 | 18.68 |
| (upper limit sample) | | | |

Results: From the results of the decrease in long-term skin water loss, the continuous use of example sample-1 for 28 days resulted in the decrease in skin water loss by 16.64%, that is, the skin water loss was constantly reduced, reflecting that the barrier function of the skin had been effectively repaired.

Compared with Comparative Example 1, Examples 1˜3 can significantly improve the moisturizing effect (long-term, instant), indicating that the composition of the present disclosure had a significant effect in the ability of moisturizing (long-term, instant); p<0.05;

Compared with Comparative Examples 2˜4, Examples 1˜3 had more significant effects in moisturizing (long-term, instant), indicating that the composition of the present disclosure was more reasonable in composition, each component was indispensable, and they cooperated with each other to produce a significant synergistic effect;

Compared with Comparative Example 5, Examples 1˜3 had more significant effects in moisturizing (long-term, instant), indicating that the composition of the present disclosure has a better effect in the same content of active ingredients, indicating that the composition of the present disclosure had a reasonable and appropriate ratio.

Among the samples, the effect of Example 3 was the most significant, indicating that the ratio and concentration were the most suitable. The effect of the obtained sample was significantly different from that of the other groups, p<0.05.

In summary, the composition for regulating biorhythm can effectively increase the energy of skin cells by 132.24%, and the increase in cell energy was reflected in the effective improvement of the skin's barrier. After 28 days of use, the skin moisture content was increased, by 17.27%, and the skin water loss was decreased, by 16.64%.

The above are only the preferred embodiments of the present disclosure. It should be pointed out that those of ordinary skill in the art may make various improvements and modifications without departing from the principle of the present disclosure, and these improvements and modifications should also be considered as the protection scope of the present disclosure.

1. A composition comprising the following components in parts by mass:

| | |
|---|---|
| skin conditioner A | 0.2~1.0 parts; |
| skin conditioner B | 0.2~2.0 parts; |
| adenosine | 0.1~2.0 parts; |

wherein the skin conditioner A comprises the following in mass fraction:

| | |
|---|---|
| yeast glycoprotein | 3%; |
| glutamic acid | 3%; |
| valine | 0.55% |
| threonine | 0.55% |
| preservative | 1.1%; |
| stabilizer | 0.05% |
| and the balance is water; | |

the skin conditioner B comprises the following in mass fraction:

| | |
|---|---|
| glutamyl amidoethyl imidazole | 1% |
| preservative | 0.4% |
| and the balance is water. | |

2. The composition according to claim 1, wherein,
the preservative in the skin conditioner A is phenoxyethanol and ethylhexylglycerol, wherein a mass ratio of phenoxyethanol and ethylhexylglycerol is 1:1;
the stabilizer in the skin conditioner A is sodium metabisulfite;
the preservative in the skin conditioner B is sorbic acid and phenoxyethanol, wherein a mass ratio of sorbic acid and phenoxyethanol is 1:3.

3. A method for regulating skin biorhythm, comprising using the composition according to claim 1.

4. A method for moisturizing and hydrating skin and promoting cell energy synthesis, comprising using the composition according to claim 1.

5. A skin care product for regulating skin biorhythm, comprising the composition according to claim 1.

6. The skin care product according to claim 5, wherein a mass fraction of the composition according to claim 1 is 0.5%˜5%.

7. The skin care product according to claim 5, comprising:

| | |
|---|---|
| the composition according to claim 1[[ or 2]] | 0.5%~5% |
| humectant | 9%; |
| penetration enhancer | 1%; |
| keratin softener | 0.4%; |
| preservative | 1%; |
| and the balance is water. | |

8. The skin care product according to claim 7, wherein,
the humectant is glycerol and 1,3-butanediol, wherein a mass ratio of glycerol and 1,3-butanediol is 1:1;
the penetration enhancer is pentanediol;
the keratin softener is hydroxyethylpiperazine ethane sulfonic acid; and
the preservative is PHL.

9. The skin care product according to claim 7, consisting of the following components in mass fraction:

| | |
|---|---|
| skin conditioner A | 0.2%~1.0%; |
| skin conditioner B | 0.2%~2.0%; |
| adenosine | 0.1%~2.0%; |
| glycerol | 8%; |
| 1,3 butanediol | 1%; |
| pentanediol | 1%; |
| hydroxy ethylpiperazine ethane sulfonic acid | 0.4%; |
| PHL | 1%; |
| and the balance is water; | |

wherein the skin conditioner A comprises the following in mass fraction:

| | |
|---|---|
| yeast glycoprotein | 3%; |
| glutamic acid | 3%; |
| valine | 0.55% |
| threonine | 0.55% |
| preservative | 1.1%; |
| stabilizer | 0.05% |
| and the balance is water; | |

the skin conditioner B comprises the following in mass fraction:

| | |
|---|---|
| glutamyl amidoethyl imidazole | 1% |
| preservative | 0.4% |
| and the balance is water. | |

10. A method for preparing the skin care product according to claim 5, comprising:
adding hydroxyethylpiperazine ethane sulfonic acid and adenosine to deionized water, heating to 75° C.˜80° C., and stirring to dissolve at 250˜300 r/min for 20 min;
adding glycerol, 1,3-butanediol and pentanediol, controlling a temperature at 65˜75° C., and stirring to dissolve at 300 r/min for 15 min; and
adding the skin conditioner A, the skin conditioner B and PHL, controlling a temperature at 35˜45° C., stirring at 100 r/min for 15 min, and then cooling to room temperature to prepare a skin care product.
